(19) United States
(12) Patent Application Publication
(10) Pub. No.: US 2026/0215782 A1
(43) Pub. Date: Jul. 30, 2026

(54) VENOUS OCCLUDER

(71) Applicant: ZHEJIANG UNIVERSITY, Zhejiang (CN)

(72) Inventors: Xin YU, Zhejiang (CN); Hong YU, Zhejiang (CN); Hongyuan LV, Zhejiang (CN); Bo SHEN, Zhejiang (CN); Zihao HUANG, Zhejiang (CN)

(73) Assignee: ZHEJIANG UNIVERSITY, Zhejiang (CN)

(21) Appl. No.: 18/722,501

(22) PCT Filed: Oct. 30, 2023
(86) PCT No.: PCT/CN2023/127599

§ 371 Date: Jun. 20, 2024

(30) Foreign Application Priority Data
Jun. 8, 2023 (CN) 202310677459.4

(51) Int. Cl. A61B 17/12; A61B 17/00; A61M 25/00; A61M 25/01; A61M 25/09; A61M 25/10

(52) U.S. Cl. CPC A61B 17/12109; A61B 2017/00238; A61B 17/12031; A61B 17/12045; A61B 2017/12127; A61M 25/0045; A61M 25/0108; A61M 25/09; A61M 2025/105

(57) ABSTRACT

A venous occluder is provided, which includes a first occluder and a second occluder. Each of the first occluder and the second occluder includes a catheter, a guide wire, a balloon, and a handle part. The guide wire is arranged inside the catheter, and the balloon and the handle part are arranged outside the catheter. The balloon is arranged at a front end of the catheter, and the handle part is arranged at a rear end of the catheter. A diameter of the second occluder, except for the handle part, is smaller than that of the catheter of the first occluder, and the part of the second occluder, except for the handle part, can pass through the catheter of the first occluder. Meanwhile, tumor thrombus located in the inferior vena cava or the hepatic vein can be pushed into the extent of hepatectomy.

### CROSS REFERENCE TO RELATED APPLICATION

This application is a national stage application of International Patent Application No. PCT/CN 2023/127599, filed on Oct. 30, 2023, which claims the priority of Chinese Patent Application No. 202310677459.4 entitled “VENOUS OCCLUDER” filed with the Chinese Patent Office on Jun. 8, 2023, which is incorporated herein by reference in its entirety.

### TECHNICAL FIELD

The present disclosure relates to the field of medical devices, and in particular to a venous occluder.

### BACKGROUND

Hepatectomy is the optimal treatment means for primary and secondary tumors in liver. The liver receives double blood supply from hepatic artery and portal vein, and the filtered blood is transported back to inferior vena cava through three hepatic veins. The special anatomical structure determines that the liver has affluent blood flow. How to effectively control intraoperative bleeding is the key to the success of hepatectomy. The bleeding of main blood vessels and the bleeding of liver section during operation mainly depends on the technique of hepatic vascular exclusion. Total hepatic vascular exclusion (Pringle method) or regional hepatic vascular exclusion is the most common hepatic vascular exclusion method in hepatectomy at present, which can effectively and completely or partially occlude the blood flow of the hepatic artery and portal vein. Hepatic vein blood flow exclusion can be combined with Pringle method to achieve total hepatic/hemi-hepatic blood flow blocking, so as to prevent blood backflow from the inferior vena cava to hepatic venous system, especially in the resection of the tumor involving the second hepatic portal, which can reduce tumor cell exfoliation and hepatic vein metastasis caused by intraoperative extrusion. The right hepatic vein, the middle hepatic vein and the left hepatic vein are generally dissected and separated, and the bleeding of the hepatic venous system is reduced by introducing a right hepatic vein blocking belt, and a common blocking belt of the middle hepatic vein and the left hepatic vein, and reducing central venous pressure or properly increasing pneumoperitoneum pressure in cooperation with anesthesiologists. A few scholars in China have developed various first hepatic portal occluders and inferior vena cava occluders for laparoscopic hepatectomy, but the methods are not yet mature, and there is also a lack of effective devices to block the hepatic venous system.

### SUMMARY

In view of this, a venous occluder is provided by the present disclosure, which is helpful for an operator to conduct effective hepatic venous or inferior vena cava blood flow blocking under laparoscopy, so as to reduce the bleeding during laparoscopic hepatectomy, reduce the possibility of switching from laparoscopic surgery to open surgery, and ensure the safety of a patient. Meanwhile, tumor thrombus located in the inferior vena cava or the hepatic vein can be pushed into the extent of hepatectomy, so as to reduce the possibility of vascular metastasis of the tumor thrombus caused by surgical operation and improve the efficiency, effect and safety of laparoscopic treatment.

A venous occluder is provided, including a first occluder, and a second occluder, wherein each of the first occluder and the second occluder comprises a catheter, a guide wire, a balloon, and a handle part; the guide wire is arranged inside the catheter, and the balloon and the handle part are arranged outside the catheter; the balloon is arranged at a front end of the catheter, and the handle part is arranged at a rear end of the catheter; and a diameter of the second occluder, except for the handle part, is smaller than that of the catheter of the first occluder. A part of the second occluder except the handle part can pass through the catheter of the first occluder.

Further, the catheter is a cylindrical hollow catheter, including a bent part and a horizontal part. The bent part is located at a front end of the catheter, and a remaining part of the catheter, except for the bent part, is the horizontal part. The balloon is arranged at one end, adjacent to the bent part, of the horizontal part, and the handle part is arranged at one end, away from the bent part, of the horizontal part.

Further, the catheter is internally provided with a first chamber, a second chamber, and a third chamber. The first chamber is located in the middle part of the catheter and is an access channel of the guide wire, the second chamber is located at the bottom of the catheter and communicates with the balloon, and the third chamber is located at the top of the catheter.

Further, the venous occluder also includes a steel wire, and a rotary hub. The steel wire is located in the third chamber and fixedly connected to the third chamber. The rotary hub is arranged outside the horizontal part, and located at a position, adjacent to the handle part, of the horizontal part, the steel wire is connected to the rotary hub, and the rotary hub is of a collar structure.

Further, the bent part is made of an elastic material, and has a smooth outer wall. The bent part can form an angle with the horizontal part under the traction of the steel wire, and the bent part can automatically reset when there is no traction force.

Further, an outer wall of the second chamber at a joint of the second chamber and the balloon is provided with multiple balloon openings, the balloon openings are circular and are uniformly arranged in a length direction of the first chamber, so that normal saline can enter the balloon from the second chamber through the balloon openings.

Further, each of a top end and a bottom end of the balloon is provided with an X-ray marker for determining a position of the balloon, and the outside of the balloon is coated with an anticoagulant drug coating for preventing blood coagulation.

Further, the guide wire is made of a medical titanium alloy material, and has a length greater than that of the catheter, and the guide wire is able to enter the first chamber from the handle part.

Further, the handle part includes a handle, a balloon port, and a guide wire inlet. The handle is arranged at the periphery of the horizontal part, and is located at one end, away from the bent part, of the horizontal part, and the balloon port and the guide wire inlet are both located at a rear end of the handle.

Further, the balloon port communicates with the second chamber, and is externally provided with a screw cap, the screw cap is configured for being connected to a nipple of a syringe; the guide wire inlet communicates with the first chamber, is configured for allowing the guide wire and the second occluder to enter, and is externally provided with a further screw cap.

Compared with the prior art, the present disclosure has the following beneficial effects:

A venous occluder provided by some embodiments is scientific and reasonable in structure, safe and convenient to operate, humanized in design, and capable of providing effective hepatic venous blood flow blocking, reducing the bleeding during laparoscopic hepatectomy, reducing the possibility of switching from laparoscopic surgery to open surgery, and ensuring the safety of a patient. Meanwhile, tumor thrombus located in the inferior vena cava or the hepatic vein can be pushed into the extent of hepatectomy, so as to reduce the possibility of vascular metastasis of the tumor thrombus caused by surgical operation and improve the efficiency, effect and safety of laparoscopic treatment. The venous occluder has broad clinical prospects.

### BRIEF DESCRIPTION OF THE DRAWINGS

FIG. 1 is a diagram of collaboration of a first occluder and a second occluder according to the present disclosure;

FIG. 2 is a structural schematic diagram of a venous occluder according to the present disclosure;

FIG. 3 is a sectional view of a venous occluder according to the present disclosure;

FIG. 4 is a front view of a venous occluder in the liver according to the present disclosure.

In the drawings: 10 first occluder; 11 catheter; 111 bent part; 112 horizontal part; 113 first chamber; 114 second chamber; 115 third chamber; 12 guide wire; 13 balloon; 131 balloon opening; 14 handle part; 141 handle; 142 balloon port; 143 guide wire inlet; 15 steel wire; 16 rotary hub; 20 second occluder; 30 inferior vena cava; 40 hepatic vein; 41 right hepatic vein; 42 middle hepatic vein; 43 left hepatic vein.

### DETAILED DESCRIPTION OF THE EMBODIMENTS

The embodiments of the present disclosure are further described below in detail with reference to accompanying drawings and embodiments. The following embodiments are used to illustrate the present disclosure rather than limiting the scope of the present disclosure.

In the description of the present disclosure, it needs to be understood that the orientation or positional relationship indicated in this description is based on the orientation or positional relationship shown in the drawings only for convenience of description of the present disclosure and simplification of description rather than indicating or implying that the device or element referred to must have a particular orientation, be configured and operate in a particular orientation, and thus can not to be construed as limiting the present disclosure.

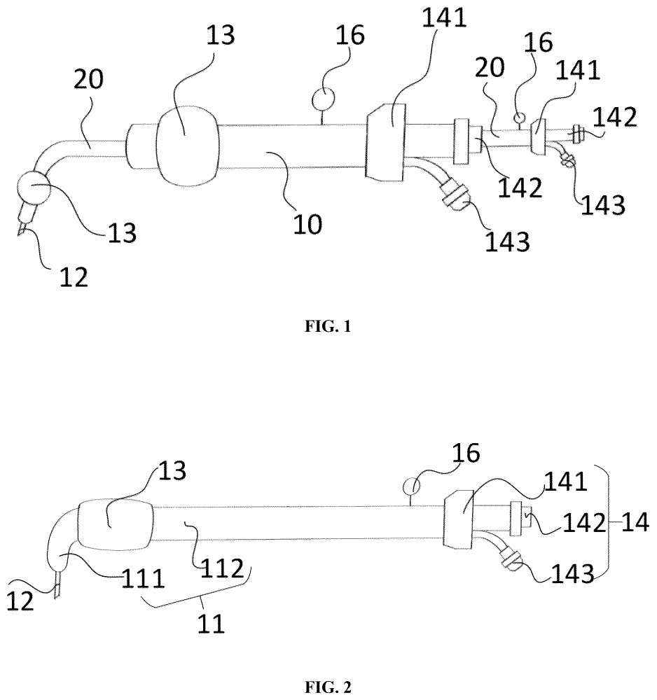

Please referring to FIG. 1 to FIG. 2, a venous occluder is provided, which includes a first occluder 10, and a second occluder 20. Each of the first occluder 10 and the second occluder 20 includes a catheter 11, a guide wire 12, a balloon 13, and a handle part 14. The guide wire 12 is arranged inside the catheter 11, and the balloon 13 and the handle part 14 are arranged outside the catheter 11. The balloon 13 is arranged at a front end of the catheter 11, and the handle part 14 is arranged at a rear end of the catheter 11. A diameter of the second occluder 20, except for the handle part 14, is smaller than that of the catheter 11 of the first occluder 10, and a part of the second occluder 20, except for the handle part 14, can pass through the catheter 11 of the first occluder 10 for operation as required. The first occluder 10 provided by the present disclosure is an inferior vena cava occluder, and the second occluder 20 is a hepatic venous occluder. A venous occluder provided by the present disclosure is scientific and reasonable in structure, safe and convenient to operate, humanized in design, and capable of providing effective hepatic venous blood flow blocking, reducing the bleeding during laparoscopic hepatectomy, reducing the possibility of switching from laparoscopic surgery to open surgery, and ensuring the safety of a patient. Meanwhile, tumor thrombus located in the inferior vena cava or the hepatic vein can be pushed into the extent of hepatectomy, so as to reduce the possibility of vascular metastasis of the tumor thrombus caused by surgical operation and improve the efficiency, effect and safety of laparoscopic treatment.

Further, the catheter 11 is a cylindrical hollow catheter, a diameter of the catheter is substantially smaller than an inner diameter of an intravenous lumen of a patient, so as to facilitate the catheter to enter the intravenous lumen of the patient. The catheter 11 is made of a medical material with a certain strength, and thus has a certain hardness and flexibility, which can not only facilitate the operation, but also avoid injuring the intravenous lumen of the patient. The catheter 11 includes a bent part 111, and a horizontal part 112. The bent part 111 is located at a front end of the catheter 11, and is made of an elastic material. Under the control of an external force, the bending degree of the bent part 111 can be adjusted to guide the occluder to enter the intravenous lumen of the patient. An outer wall of the bent part 111 is smooth and flexible, thus avoiding injuring blood vessels. The remaining part of the catheter, except for the bent part 111, is the horizontal part 112, the balloon 13 is arranged at one end, adjacent to the bent part 111, of the horizontal part 112, and the handle part 14 is arranged at one end, away from the bent part 111, of the horizontal part 112.

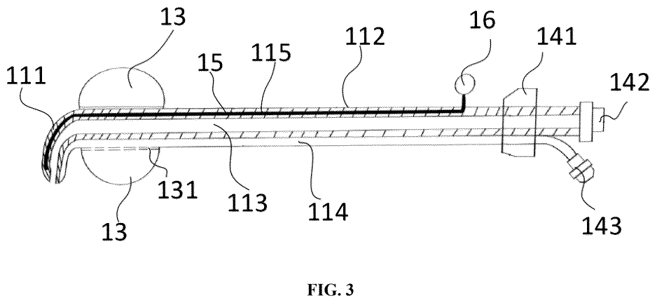

Please referring to FIG. 3, the catheter 11 is internally provided with a first chamber 113, a second chamber 114, and a third chamber 115.

Further, the first chamber 113 is a guide wire chamber, which is located in the middle of the catheter 11 and is a channel for the guide wire to enter or exit the occluder. The guide wire 12 is made of a medical titanium alloy material, and has a length greater than that of the catheter 11. The guide wire 12 can enter the first chamber 113 from the handle part 14 and exit from the foremost end of the catheter 11, and is configured to provide a certain hardness for the catheter 11 and guide the first occluder 10 and the second occluder 20 to reach a designated vascular site from a superficial vein entrance.

Further, the second chamber 114 located at the bottom of the catheter 11 is a chamber for the balloon 13. The second chamber 114 communicates with the balloon 13, and is a normal saline injection channel. The normal saline, after entering the second chamber 114, enters the balloon 13. The balloon 13 may be made of a medical silicon material or other materials, and has a certain tension after being injected with the normal saline. When the balloon 13 is dilated to have a diameter equivalent to the inner diameter of the vein, the blood flow there can be blocked, or the balloon 13 can be used as a baffle to push the tumor thrombus in the vein. X-ray markers are arranged at dilated ends of the balloon 13 to determine the position of the balloon 13, which is convenient for imaging or esophageal ultrasonic positioning. The balloon 13 is coated with an anticoagulant drug to prevent blood coagulation.

Specifically, an outer wall of the second chamber 114 at a joint of the second chamber 114 and the balloon 13 is provided with multiple balloon openings 131, the balloon openings 131 are circular and are uniformly arranged in a length direction of the first chamber 114, and thus normal saline can enter the balloon 13 from the second chamber 114 through the balloon openings 131 to dilate the balloon 13.

Further, the venous occluder according to the present disclosure further includes a steel wire 15. The third chamber 115 is a chamber for the steel wire and is fixedly connected to the steel wire 15. The bent part 111 may form a certain angle with the horizontal part 112 under the traction of the steel wire 15, a value of the angle is decided by the traction force of the steel wire 15, and the bent part 111 can automatically reset when there is no traction force. A rotary hub 16 is arranged at a position, adjacent to the handle part 14, of the horizontal part 112. The rotary hub 16 is a collar with a diameter allowing a finger of an operator to pass through. The rotary hub 16 is connected to the steel wire 15. The operator wears the collar with the finger, and rotates the rotary hub 16 to pull the steel wire 15. The rotation angle of the rotary hub 16 determines bending degree and direction of the bent part 111.

Further, the handle part 14 includes a handle 141, a balloon port 142, and a guide wire inlet 143. The handle 141 is arranged at the periphery of the horizontal part 112, and is located at one end, away from the bent part 111, of the horizontal part 112. The balloon port 142 and the guide wire inlet 143 are both located at a rear end of the handle 141. The handle 141 provides a handle part for the operator during operation. The balloon port 142 is obtained by extending the second chamber 114, and is externally covered with a screw cap, which is configured for being connected to a nipple of a syringe to inject the normal saline. The guide wire inlet 143 is obtained by extending the first chamber 113 for the guide wire 12 and the second occluder 20 to pass through, and is externally covered with a further screw cap.

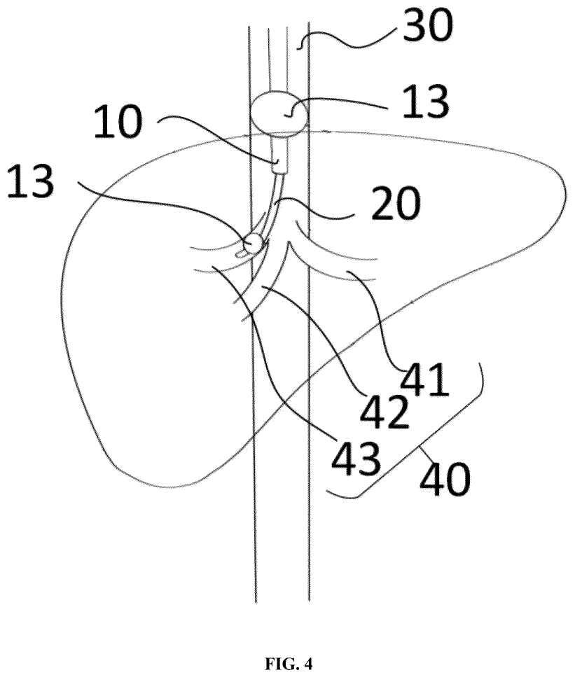

Please referring to FIG. 4, a venous occluder provided by the present disclosure has two working paths specifically as follows.

The balloon 13 of the first occluder is sent from a right internal jugular vein to corresponding inferior vena cava 30 for blood flow blocking through superior vena cava, atrium dextrum and inferior vena cava 30, or sent from femoral vein to corresponding inferior vena cava 30 for blood flow blocking through external iliac vein and internal iliac vein. After the balloon 13 of the first occluder is fixed, the balloon 13 of the second occluder 20 is sent to corresponding hepatic vein 40 for hepatic vein blood flow blocking through the first chamber 113 of the first occluder 10.

Further, a venous occluder provided by the present disclosure has four usage modes specifically as follows.

1. The right internal jugular vein is disinfected, and then an incision is made with a scalpel. After a vascular catheter is used for dilation, the first occluder 10 provided the present disclosure is guided to the inferior vena cava 30. If there is no tumor thrombus in the inferior vena cava 30, normal saline is injected to dilate the balloon 13, so as to fix the balloon 13. Whether a position of the balloon 13 is correct or not is determined by means of esophageal ultrasound examination. If the position of the balloon 13 is correct, the normal saline is injected to further dilate the balloon 13 to block the blood flow in the inferior vena cava 30, and the guide wire 12 is withdrawn.

2. If the operation only involves a certain hepatic lobe, the first occluder 10 provided by the present disclosure can be guided to one of the right hepatic vein 41, the middle hepatic vein 42 and the left hepatic vein 43. Because the hepatic vein 40 merges into the inferior vena cava 30, and there is an included angle between the hepatic vein 40 and the inferior vena cava 30. It is necessary to control the bent part 111 by the rotary hub 16, so as to make the balloon 13 smoothly enter the target hepatic vein 40 through the included angle.

3. If the operation involves a large extent of hepatectomy, and the possibility of bleeding is high, the first occluder 10 provided by the present disclosure can be guided to the inferior vena cava 30. If there is no tumor thrombus in the inferior vena cava 30, the normal saline is injected to dilate the balloon 13, so as to fix the balloon 13. Whether a position of the balloon 13 is correct or not is determined by means of esophageal ultrasound examination. If the position of the balloon 13 is correct, the normal saline is injected to further dilate the balloon 13 to block the blood flow in the inferior vena cava 30, and the guide wire 12 is withdrawn. The second occluder 20 is fed through the first chamber 113 in the first occluder 10, and then is guided by the guide wire 12, and the bent part 111 is controlled by the rotary hub 16, so as to make the balloon 13 smoothly enter the target hepatic vein 40 through the included angle. If the position of the balloon 13 is correct, the normal saline is injected to further dilate the balloon 13 to block the blood flow of the hepatic vein 40 there.

After the operation is finished, the normal saline in the balloon 13 is sucked, the second occluder 20 and the first occluder 10 are sequentially pulled out, and hemostasis is performed.

4. If there is a tumor thrombus in the inferior vena cava 30 or the hepatic vein 40, the first occluder 10 is guided to the front of the tumor thrombus (outside the extent of resection of surgery) through the guide wire 12, and the normal saline is injected to dilate the balloon 13. After the balloon 13 is fixed, the second occluder 20 is placed, and then the second occluder 20 is guided to pass through the tumor thrombus through the guide wire 12, and located behind the tumor thrombus (inside extent of resection of surgery). Afterwards, the normal saline is injected to dilate the balloon 13 to block the blood flow behind the tumor thrombus, so as to prevent the tumor thrombus from falling out of the extent of resection of surgery. The dilated balloon 13 in the first occluder 10 is pushed inward to push the tumor thrombus into the extent of resection of surgery, then the balloon 13 is fixed to block the blood flow of the hepatic vein 40, and then the guide wire 12 is withdrawn. The remaining steps are the same as above.

It should be noted that the venous occluder provided by the present disclosure further has a usage mode as follows.

The venous occluder is used after a vascular sheath is preset, the vascular sheath can provide a vascular access for the venous occluder provided by the present disclosure.

As can be known from above that a venous occluder provided by the present disclosure is scientific and reasonable in structure, safe and convenient to operate, humanized in design, and capable of providing effective hepatic venous blood flow blocking, reducing the bleeding during laparoscopic hepatectomy, reducing the possibility of switching from laparoscopic surgery to open surgery, and ensuring the safety of a patient. Meanwhile, tumor thrombus located in the inferior vena cava or the hepatic vein can be pushed into the extent of hepatectomy, so as to reduce the possibility of vascular metastasis of the tumor thrombus caused by surgical operation and improve the efficiency, effect and safety of laparoscopic treatment. So that the venous occluder has broad clinical prospects.

The above is only the preferred embodiment of the present disclosure, rather than any form limiting the present disclosure. Although the present disclosure has been shown in the preferred embodiment, it is not intended to limit the present disclosure. A person skilled in the art can make some equivalent variations, alterations or modifications to the above technical content without departing from the scope of the technical solutions of the present disclosure to obtain equivalent embodiments. Any simple alteration, equivalent change or modification made to the above embodiments according to the technical essence of the present disclosure without departing from the content of the technical solutions of the present disclosure shall fall within the scope of the technical solutions of the present disclosure.

What is claimed is:

1. A venous occluder, comprising a first occluder (10), and a second occluder (20), wherein each of the first occluder (10) and the second occluder (20) comprises a catheter (11), a guide wire (12), a balloon (13), and a handle part (14); the guide wire (12) is arranged inside the catheter (11), and the balloon (13) and the handle part (14) are arranged outside the catheter (11);
the balloon (13) is arranged at a front end of the catheter (11), and the handle part (14) is arranged at a rear end of the catheter (11); and a diameter of the second occluder (20), except for the handle part (14), is smaller than that of the catheter (11) of the first occluder (10).

2. The venous occluder according to claim 1, wherein the catheter (11) is a cylindrical hollow catheter, comprising a bent part (111) and a horizontal part (112); the bent part (111) is located at the front end of the catheter (11), and the remaining part of the catheter (11), except for the bent part (111), is the horizontal part (112); the balloon (13) is arranged at one end, adjacent to the bent part (111), of the horizontal part (112), and the handle part (14) is arranged at one end, away from the bent part (111), of the horizontal part (112).

3. The venous occluder according to claim 2, wherein the catheter (11) is internally provided with a first chamber (113), a second chamber (114), and a third chamber (115); the first chamber (113) is located in a middle part of the catheter (11) and is an access channel of the guide wire (12), the second chamber (114) is located at a bottom of the catheter (11) and communicates with the balloon (13), and the third chamber (115) is located at a top of the catheter (11).

4. The venous occluder according to claim 3, further comprising a steel wire (15), and a rotary hub (16), wherein the steel wire (15) is located in the third chamber (115) and fixedly connected to the third chamber (115); the rotary hub (16) is arranged outside the horizontal part (112), and located at a position, adjacent to the handle part (14), of the horizontal part (112), the steel wire (15) is connected to the rotary hub (16), and the rotary hub (16) is of a collar structure.

5. The venous occluder according to claim 4, wherein the bent part (111) is made of an elastic material, and has a smooth outer wall; the bent part (111) is able to form an angle with the horizontal part (112) under traction of the steel wire (15), and the bent part (111) is able to automatically reset when no traction force exists.

6. The venous occluder according to claim 3, wherein an outer wall of the second chamber (114) at a joint of the second chamber (114) and the balloon (13) is provided with a plurality of balloon openings (131), the balloon openings (131) are circular, the plurality of balloon openings

(131) are uniformly arranged in a length direction of the first chamber (114), so that normal saline is able to enter the balloon (13) from the second chamber (114) through the balloon openings (131).

7. The venous occluder according to claim 1, wherein each of a top end and a bottom end of the balloon (13) is provided with an X-ray marker for determining a position of the balloon (13), and an outside of the balloon (13) is coated with an anticoagulant drug coating for preventing blood coagulation.

8. The venous occluder according to claim 3, wherein the guide wire (12) is made of a medical titanium alloy material, and has a length greater than that of the catheter (11), and the guide wire (12) is able to enter the first chamber (113) from the handle part (14).

9. The venous occluder according to claim 3, wherein the handle part (14) comprises a handle (141), a balloon port (142), and a guide wire inlet (143); the handle (141) is arranged at a periphery of the horizontal part (112), and is located at one end, away from the bent part (111), of the horizontal part (112), and the balloon port (142) and the guide wire inlet (143) are both located at a rear end of the handle (141).

10. The venous occluder according to claim 9, wherein the balloon port (142) communicates with the second chamber (114), and is externally provided with a screw cap, the screw cap is configured for being connected to a nipple of a syringe; the guide wire inlet (143) communicates with the first chamber (113), is configured for allowing the guide wire (12) and the second occluder (20) to enter, and is externally provided with a further screw cap.
